(19) United States
(12) Patent Application Publication
(10) Pub. No.: US 2018/0346874 A1
(43) Pub. Date: Dec. 6, 2018

(54) METHODS OF OBTAINING CELLS FROM HUMAN POSTPARTUM UMBILICAL CORD ARTERIAL TISSUE

(71) Applicant: Janssen Biotech, Inc., Horsham, PA (US)

(72) Inventors: Ian R. Harris, Spring House, PA (US); Nadine S. Dejneka, Spring House, PA (US)

(73) Assignee: Janssen Biotech, Inc., Horsham, PA (US)

(21) Appl. No.: 15/988,782

(22) Filed: May 24, 2018

Related U.S. Application Data
(60) Provisional application No. 62/514,317, filed on Jun. 2, 2017.

(51) Int. Cl. C12N 5/073; A61K 35/51

(52) U.S. Cl. CPC C12N 5/0605; C12N 2509/00; A61K 35/51

(57) ABSTRACT

The invention provides for methods of obtaining cells from mammalian, preferably human, postpartum umbilical cord arterial tissue and use of these cells to treat ocular degenerative conditions. In particular, methods of the invention result in the isolation of a homogenous population of human postpartum umbilical cord arterial tissue-derived cells. In certain embodiments, the cells: express CD13, CD90, NG2, and HLA-ABC; do not express one or more of CD31, CD34, CD45, CD117, FSP1, and E-cadherin. In other embodiments, the cells secrete one or more of thrombospondin-1, BDNF and sVEGFR1.

### CROSS REFERENCE TO RELATED APPLICATIONS

This application claims benefit of U.S. Provisional Application Ser. No. 62/514,317, filed Jun. 2, 2017, the entire contents of which is incorporated by reference herein in its entirety.

### FIELD OF THE INVENTION

This invention relates to methods of obtaining cells from mammalian, preferably human, postpartum umbilical cord arterial tissue and use of these cells to derive cells for treating ocular degenerative conditions

### BACKGROUND OF THE INVENTION

Various protocols have been devised to obtain cells suitable for progenitor cell therapy. Of interest to those in the field have been protocols to obtain progenitor cells from readily available sources such as amniotic fluid, umbilical cord blood, postpartum umbilical cord tissue and postpartum placental tissue.

In particular, different progenitor cell populations have been obtained from umbilical cord tissue. For instance, cells suitable for cell therapy have been obtained from Wharton's Jelly in umbilical cord tissue. For example, Purchio et al. (U.S. Pat. No. 5,919,702) isolated chondrogenic progenitor cells (or prechondrocytes) from Wharton's Jelly. Cells suitable for cell therapy have also been obtained from whole umbilical cord tissue by washing umbilical cord tissue with an enzymatic cocktail. See e.g. Weiss et al., Stem Cells; 24:781-792 (2006). Alternatively, cells have been obtained from whole umbilical cord tissue using mincing in combination with enzymatic digestion. See U.S. Pat. No. 7,510,873. These cells have been proven therapeutically useful in the treatment of a variety diseases such as Alzheimer's, amyolateral sclerosis, retinitis pigmentosa and ocular degenerative conditions including age-related macular degeneration. Each of these protocols leads to the isolation of different progenitor cell populations from postpartum umbilical cord tissue having differing therapeutic potentials.

Another protocol is needed for obtaining progenitor cells from umbilical cord tissue that are therapeutically useful, in particular for the treatment of ocular degenerative conditions.

### SUMMARY OF THE INVENTION

The invention provides methods of generating human umbilical cord arterial tissue-derived cells from human umbilical cord tissue as well as uses of these cells. In particular, cells produced by the methods of the invention are suitable for treating ocular degenerative conditions.

The human umbilical cord arterial tissue-derived cells obtained by the methods of the invention may be progenitor cells. In other embodiments, the umbilical cord arterial tissue-derived cells obtained by the methods of the invention are umbilical cord arterial tissue smooth muscle-derived cells.

In certain embodiments, the cells of the invention are able to differentiate into an ocular lineage or phenotype. In other embodiments, the cells are able to differentiate into a neural lineage or phenotype. In alternate embodiments, the cells are able to differentiate into: (1) an ocular lineage or phenotype; and (2) neural lineage or phenotype.

In embodiments, the cells are capable of self-renewal and expansion in culture, have the potential to differentiate into cells of other phenotypes and further have the following characteristics: express at least one of CD10, CD13, CD90, NG2, and HLA-ABC; and do not express one or more of CD31, CD34, CD45, CD117, FSP1, E-cadherin and HLA DR, DP, DQ. In embodiments, the cells are capable of self-renewal and expansion in culture; have the potential to differentiate into cells of other phenotypes. In some embodiments, the cells express CD13, CD90, NG2, and HLA-ABC, and do not express CD31, CD34, CD45, CD117, FSP1, and E-cadherin. In embodiments, the cells express alpha smooth muscle actin (α-smooth muscle actin). In one embodiment, the cells have one or more of, or all of the characteristics identified in Table 2-1 and 2-2 in Example 2.

In certain embodiments, the cells secrete a trophic factor suitable for treating ocular degeneration. For instance, the cells of the invention secrete one or more of a synaptogenic factor or a bridge molecule. In embodiments, the cells secrete one or more of thrombospondin-1, thrombospondin-2, thrombospondin-4, RTK ligands, BDNF, HDF and soluble VEGFR1. In one embodiment, the cells secrete one or more thrombospondin-1, thrombospondin-2 or thrombospondin-4. In another embodiment, the cells secrete each of thrombospondin-1, thrombospondin-2 and thrombospondin-4. In the embodiments described or disclosed herein, the umbilical cord arterial tissue-derived cells secrete one or more of thrombospondin-1 (TSP-1), brain-derived neurotrophic factor (BDNF), and soluble VEGFR1. In the embodiments herein, the cells secrete thrombospondin-1, BDNF, and soluble VEGFR1. In the embodiments described or disclosed herein, the umbilical cord arterial tissue-derived cells have increased expression of TSP-1 compared to whole umbilical cord tissue cells, such as the human umbilical cord tissue-derived cells (hUTC) isolated in U.S. Pat. No. 7,510,873.

One embodiment of the invention is a method for generating human umbilical cord arterial tissue-derived cells from human umbilical cord tissue involving: (a) obtaining umbilical cord tissue; (b) removing substantially all of the blood from the tissue to yield umbilical tissue substantially free of blood; (c) separating human umbilical cord arterial tissue from the umbilical cord tissue; (d) dissociating the human umbilical cord arterial tissue substantially free of blood by mechanical dissociation such as mincing; (e) digesting the dissociated tissue with a mixture of enzymes comprising a metalloprotease, neutral protease and mucolytic enzyme; (f) isolating the umbilical cord arterial tissue-derived cells from the digested tissue; and (g) culturing the cells to obtain a homogenous population of isolated umbilical cord arterial tissue-derived cells. In another embodiment, the method for generating human umbilical cord arterial tissue-derived cells from human umbilical cord tissue involves: (a) obtaining human umbilical cord arterial tissue; (b) removing substantially all of the blood from the tissue to yield umbilical tissue substantially free of blood; (c) dissociating the human umbilical cord arterial tissue substantially free of blood by mechanical dissociation such as mincing; (d) digesting the dissociated tissue with a mixture of enzymes comprising a metalloprotease, neutral protease and mucolytic enzyme; (e) isolating the umbilical cord arterial tissue-derived cells from the digested tissue; and (f) culturing the cells to obtain a homogenous population of isolated umbilical cord arterial tissue-derived cells. The methods of the invention also include separating umbilical cord arterial tissue from Wharton's Jelly.

In another embodiment, the invention provides a method for generating human umbilical cord arterial tissue-derived cells comprising: (a) dissociating human umbilical cord arterial tissue substantially free of blood by mechanical dissociation such as mincing; (b) digesting the dissociated tissue with a mixture of enzymes comprising a metalloprotease, neutral protease and mucolytic enzyme; (c) isolating umbilical cord arterial tissue-derived cells from the digested tissue; (d) resuspending the isolated cells in a growth medium; and (e) culturing the isolated cells to obtain a homogenous population of isolated cells.

In certain embodiments, these methods also include suspending the cells in a growth medium after isolation. They also include expanding the population of isolated umbilical cord arterial tissue-derived cells in culture, for example, for about 10 days, to confluence.

The isolated cells are cultured under conditions sufficient to generate a homogeneous population of isolated umbilical cord arterial tissue-derived cells. For example, the isolated cells may be cultured for about 10 to about 100 hours to obtain a homogenous population of isolated umbilical cord arterial tissue-derived cells.

The step of culturing to obtain a homogeneous population of cells may also include selecting for cells that express CD10. As such, in certain embodiments, the invention provides for generating human umbilical cord arterial tissue-derived cells including: (a) dissociating human umbilical cord arterial tissue substantially free of blood by mechanical dissociation such as mincing; (b) digesting the dissociated tissue with a mixture of enzymes comprising a metalloprotease, neutral protease and mucolytic enzyme; (c) isolating umbilical cord arterial tissue-derived cells from the digested tissue; (d) resuspending the isolated cells in a growth medium; and (e) culturing and selecting the isolated cells for cells that express CD10 to obtain a homogenous population of isolated cells.

In preferred embodiments, the dissociation is carried out by mincing the tissue. The removal of substantially all of the blood (or all of the blood) includes removal of free or clotted blood by one or more of washing, suctioning, blotting, centrifugal separation, or enzymatic removal. In certain embodiments of the invention, all of the blood is removed prior to dissociation.

The digestion step includes incubating the dissociated tissue with the mixture of enzymes for one or more hours. In certain embodiments, the digestion step comprises incubating the dissociated tissue with the mixture of enzymes at about 37° C. In certain embodiments, the metalloprotease enzyme is collagenase, the neutral protease enzyme is dispase, the mucolytic enzyme is hyaluronidase or combinations thereof. In certain embodiments, the methods of the invention require digesting dissociated tissue with a mixture of collagenase, dispase and hyaluronidase.

An embodiment of the invention is a method of treating an ocular degenerative condition comprising administering to a subject umbilical cord arterial tissue-derived cells obtained by the methods described above or disclosed herein. Another embodiment is use of umbilical cord arterial tissue-derived cells obtained by the methods described above or disclosed herein for treating an ocular degenerative condition. One embodiment is a composition comprising umbilical cord arterial tissue-derived cells obtained by the methods described above or disclosed herein for use in treating an ocular degenerative condition. In the embodiments described above or disclosed herein, a conditioned medium prepared from the umbilical cord arterial tissue-derived cells obtained by the methods described above or disclosed herein may be used for treating an ocular degenerative condition.

### DETAILED DESCRIPTION

Various patents and other publications are referred to throughout the specification. Each of these publications is incorporated by reference herein, in its entirety. In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense.

### 1. Definitions

Stem cells are undifferentiated cells defined by the ability of a single cell both to self-renew and to differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation, and to contribute substantially to most, if not all, tissues following injection into blastocysts. Stem cells are classified according to their developmental potential as: (1) totipotent; (2) pluripotent; (3) multipotent; (4) oligopotent; and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood). Cells that are oligopotent can give rise to a more restricted subset of cell lineages than multipotent stem cells; and cells that are unipotent are able to give rise to a single cell lineage (e.g., spermatogenic stem cells). Stem cells are also categorized based on the source from which they may be obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. Induced pluripotent stem cells (iPS cells) are adult cells that are converted into pluripotent stem cells. (Takahashi et al., Cell, 2006; 126(4):663-676; Takahashi et al., Cell, 2007; 131:1-12). An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development). Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation. In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types.

This type of progenitor cell is generally not able to self-renew. Accordingly, if this type of cell is referred to herein, it will be referred to as a non-renewing progenitor cell or as an intermediate progenitor or precursor cell.

As used herein, the phrase “differentiates into an ocular lineage or phenotype” refers to a cell that becomes partially or fully committed to a specific ocular phenotype, including without limitation, retinal and corneal stem cells, pigment epithelial cells of the retina and iris, photoreceptors, retinal ganglia and other optic neural lineages (e.g., retinal glia, microglia, astrocytes, Mueller cells), cells forming the crystalline lens, and epithelial cells of the sclera, cornea, limbus and conjunctiva. The phrase “differentiates into a neural lineage or phenotype” refers to a cell that becomes partially or fully committed to a specific neural phenotype of the CNS or PNS, i.e., a neuron or a glial cell, the latter category including without limitation astrocytes, oligodendrocytes, Schwann cells and microglia.

The cells exemplified herein and preferred for use in the present invention are generally referred to as postpartum umbilical cord arterial tissue-derived cells. They also may be described as being stem or progenitor cells, the latter term being used in the broad sense. The term derived is used to indicate that the cells have been obtained from their biological source and grown or otherwise manipulated in vitro (e.g., cultured in a growth medium to expand the population and/or to produce a cell line). The in vitro manipulations of umbilical cord arterial tissue-derived cells and their unique features are described in detail below.

Various terms are used to describe cells in culture. Cell culture refers generally to cells taken from a living organism and grown under controlled conditions (“in culture” or “cultured”). A primary cell culture is a culture of cells, tissues or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore, the number of population doublings of a culture is greater than the passage number. The expansion of cells (i.e., the number of population doublings) during the period between passaging depends on many factors, including but not limited to the seeding density, substrate, medium, growth conditions, and time between passaging.

The term “growth medium” generally refers to a medium sufficient for the culturing of the cells of the invention. In particular, one medium for the culturing of the cells of the invention in comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics ((preferably 50-100 Units/milliliter penicillin, 50-100 microgram/milliliter streptomycin, and 0-0.25 microgram/milliliter amphotericin B; Invitrogen, Carlsbad, Calif.)), and 0.001% (v/v) 2-mercaptoethanol (Sigma, St. Louis Mo.). As used in the Examples below, growth medium refers to DMEM-low glucose with 15% fetal bovine serum and antibiotics/antimycotics (when penicillin/streptomycin are included, it is preferably at 50 U/ml and 50 microgram/ml respectively; when penicillin/streptomycin/amphotericin are used, it is preferably at 100 U/ml, 100 microgram/ml and 0.25 microgram/ml, respectively). In some cases, different growth media are used, such as smooth muscle basal medium (with or without serum), or different supplementations are provided, and these are normally indicated in the text as supplementations to growth medium.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies and granules. The medium containing the cellular factors is the conditioned medium.

Generally, a trophic factor is defined as a substance that promotes survival, growth, differentiation, proliferation and/or maturation of a cell, or stimulates increased activity of a cell. The interaction between cells via trophic factors may occur between cells of different types. Cell interaction by way of trophic factors is found in essentially all cell types, and is a particularly significant means of communication among neural cell types. Trophic factors also can function in an autocrine fashion, i.e., a cell may produce trophic factors that affect its own survival, growth, differentiation, proliferation and/or maturation.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone.

The terms ocular, ophthalmic and optic are used interchangeably herein to define “of, or about, or related to the eye.” The term ocular degenerative condition (or disorder) is an inclusive term encompassing acute and chronic conditions, disorders or diseases of the eye, inclusive of the neural connection between the eye and the brain, involving cell damage, degeneration or loss. An ocular degenerative condition may be age-related, or it may result from injury or trauma, or it may be related to a specific disease or disorder. Acute ocular degenerative conditions include, but are not limited to, conditions associated with cell death or compromise affecting the eye including conditions arising from cerebrovascular insufficiency, focal or diffuse brain trauma, diffuse brain damage, infection or inflammatory conditions of the eye, retinal tearing or detachment, intra-ocular lesions (contusion penetration, compression, laceration) or other physical injury (e.g., physical or chemical burns). Chronic ocular degenerative conditions (including progressive conditions) include, but are not limited to, retinopathies and other retinal/macular disorders such as retinitis pigmentosa (RP), age-related macular degeneration (AMD), choroidal neovascular membrane (CNVM); retinopathies such as diabetic retinopathy, occlusive retinopathy, sickle cell retinopathy and hypertensive retinopathy, central retinal vein occlusion, stenosis of the carotid artery, optic neuropathies such as glaucoma and related syndromes; disorders of the lens and outer eye, e.g., limbal stem cell deficiency (LSCD), also referred to as limbal epithelial cell deficiency (LECD), such as occurs in chemical or thermal injury, Steven-Johnson syndrome, contact lens-induced keratopathy, ocular cicatricial pemphigoid, congenital diseases of aniridia or ectodermal dysplasia, and multiple endocrine deficiency-associated keratitis.

The term treating (or treatment of) an ocular degenerative condition refers to ameliorating the effects of, or delaying, halting or reversing the progress of, or delaying or preventing the onset of, an ocular degenerative condition as defined herein.

The term effective amount refers to a concentration or amount of a reagent or pharmaceutical composition, such as a growth factor, differentiation agent, trophic factor, cell population or other agent, that is effective for producing an intended result, including cell growth and/or differentiation in vitro or in vivo, or treatment of ocular degenerative conditions, as described herein. With respect to growth factors, an effective amount may range from about 1 nanogram/milliliter to about 1 microgram/milliliter. With respect to cells as administered to a patient in vivo, an effective amount may range from as few as several hundred or fewer, to as many as several million or more. In specific embodiments, an effective amount may range from 10³ to 11¹¹, more specifically at least about 10⁴ cells. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or pharmaceutical composition to achieve its intended result.

The term patient or subject refers to animals, including mammals, preferably humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

Several terms are used herein with respect to cell replacement therapy. The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy. Transplantation as used herein refers to the introduction of autologous, or allogeneic donor cell replacement therapy into a recipient.

As used herein, the term “about” when referring to a measurable value such as an amount, a temporal duration, and the like, is meant to encompass variations of between ±20% and ±0.1%, preferably ±20% or ±10%, more preferably ±5%, even more preferably ±1%, and still more preferably ±0.1% from the specified value, as such variations are appropriate to perform the disclosed methods.

### 2. Derivation of Cells from Postpartum Umbilical Cord Arterial Tissue

The invention provides for methods of deriving cells from postpartum umbilical cord arterial tissue (e.g. arterial smooth muscle tissue). To derive these cells, a mammalian umbilical cord is recovered upon or shortly after termination of either a full-term or pre-term pregnancy, for example, after expulsion of after-birth. In certain embodiments, the umbilical cord may be obtained from pigs, rats or mice. In other, more preferred embodiments, human umbilical cord is used.

The postpartum tissue may be transported from the birth site to a laboratory in a sterile container such as a flask, beaker, culture dish or bag. The container may have a solution or medium, including but not limited to a salt solution, such as, for example, Dulbecco's Modified Eagle's Medium (DMEM) or phosphate buffered saline (PBS), or any solution used for transportation of organs used for transplantation, such as University of Wisconsin solution or perfluorochemical solution. One or more antibiotic and/or antimycotic agents, such as but not limited to penicillin, streptomycin, amphotericin B, gentamicin, and nystatin, may be added to the medium or buffer. The postpartum tissue may be rinsed with an anticoagulant solution such as heparin-containing solution. It is preferable to keep the tissue at about 4-10° C. prior to extraction and derivation of the postpartum umbilical cord arterial tissue-derived cells. It is even more preferable that the tissue not be frozen prior to extraction of the umbilical arterial tissue-derived cells.

Derivation of the umbilical artery smooth muscle tissue-derived cells preferably occurs in an aseptic environment. The umbilical cord may be separated from the placenta by means known in the art. Alternatively, the umbilical cord and placenta are used without separation. Blood and debris are preferably removed from the postpartum tissue prior to isolation of the cells. For example, the postpartum tissue may be washed with buffer solution, such as but not limited to phosphate buffered saline. The wash buffer also may comprise one or more antimycotic and/or antibiotic agents, such as but not limited to penicillin, streptomycin, amphotericin B, gentamicin and nystatin.

After washing, the umbilical cord arterial tissue (including arterial smooth muscle tissue) is separated from the umbilical cord tissue (or the umbilical cord tissue and placental tissue) by means in the art. Alternatively, the umbilical cord arterial tissue (including arterial smooth muscle tissue) is separated from the umbilical cord tissue (or the umbilical cord tissue and placental tissue) prior to removal of blood and debris. In particular, the umbilical cord arterial wall is separated from Wharton's Jelly such that no Wharton's Jelly cells are isolated.

Postpartum tissue comprising umbilical cord arterial tissue (including arterial smooth muscle tissue), or a fragment or section thereof is disaggregated by mechanical force (mincing or shear forces). In a presently preferred embodiment, the isolation procedure also utilizes an enzymatic digestion process. Many enzymes are known in the art to be useful for the isolation of individual cells from complex tissue matrices to facilitate growth in culture. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single stranded DNA and can minimize cell clumping during isolation. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods that employ digestion in the presence of at least one collagenase from Clostridium histolyticum, and either of the protease activities, dispase and thermolysin. Still more preferred are methods employing digestion with both collagenase and dispase enzyme activities. Also preferred are methods that include digestion with a hyaluronidase activity in addition to collagenase and dispase activities. The skilled artisan will appreciate that many such enzyme treatments are known in the art for isolating cells from various tissue sources. For example, the LIBERASE™ Blendzyme 3 (Roche) series of enzyme combinations are suitable for use in the instant methods. Other sources of enzymes are known, and the skilled artisan may also obtain such enzymes directly from their natural sources. The skilled artisan is also well equipped to assess new or additional enzymes or enzyme combinations for their utility in isolating the cells of the invention.

The enzyme treatments are 0.5, 1, 1.5, or 2 hours long or longer. In one embodiment, the enzyme treatment lasts from about 0.5 hours to about 2 hours, alternatively from about 0.5 to about 2.5 hours, alternatively from about 1 hour to about 3 hours, alternatively from about 1.5 to about 2.5 hours. In other embodiments, the tissue is incubated at 37° C. during the enzyme treatment of the dissociation step.

The isolated cells are transferred to sterile tissue culture vessels either uncoated or coated with extracellular matrix or ligands such as laminin, collagen (native, denatured or crosslinked), gelatin, fibronectin, and other extracellular matrix proteins. The cells are cultured in any culture medium capable of sustaining growth of the cells such as, but not limited to, DMEM (high or low glucose), advanced DMEM, DMEM/MCDB 201, Eagle's basal medium, Ham's F10 medium (F10), Ham's F-12 medium (F12), Iscove's modified Dulbecco's medium, Mesenchymal Stem Cell Growth Medium (MSCGM), DMEM/F12, RPMI 1640, and Cellgro FREE™. The culture medium may be supplemented with one or more components including, for example, fetal bovine serum (FBS), preferably about 2-15% (v/v); equine serum (ES); human serum (HS); beta-mercaptoethanol (BME or 2-ME), preferably about 0.001% (v/v); one or more growth factors, for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF) and erythropoietin; amino acids, including L-valine; and one or more antibiotic and/or antimycotic agents to control microbial contamination, such as, for example, penicillin G, streptomycin sulfate, amphotericin B, gentamicin, and nystatin, either alone or in combination. In certain embodiments, the culture medium comprises growth medium (DMEM-low glucose, serum, BME, and an antibiotic agent). The cells are seeded in culture vessels at a density to allow cell growth. In one embodiment, the cells are cultured at about 0 to about 5 percent by volume CO₂ in air. In some embodiments, the cells are cultured at about 2 to about 25 percent O₂ in air, preferably about 5 to about 20 percent O₂ in air. The cells may be cultured at about 25 to about 40° C., more preferably at about 37° C. The cells are preferably cultured in an incubator. The medium in the culture vessel can be static or agitated, for example, using a bioreactor. The umbilical cord arterial tissue-derived cells preferably are grown under low oxidative stress (e.g., with addition of glutathione, Vitamin C, Catalase, Vitamin E, N-Acetylcysteine). “Low oxidative stress,” as used herein, refers to conditions of no or minimal free radical damage to the cultured cells.

Methods for the selection of the most appropriate culture medium, medium preparation, and cell culture techniques are well known in the art and are described in a variety of sources, including Doyle et al., (eds.), 1995, CELL & TISSUE CULTURE: LABORATORY PROCEDURES, John Wiley & Sons, Chichester; and Ho and Wang (eds.), 1991, ANIMAL CELL BIOREACTORS, Butterworth-Heinemann, Boston, which are incorporated herein by reference.

In some embodiments of the invention, the umbilical cord arterial tissue-derived cells are passaged, or removed to a separate culture vessel containing fresh medium of the same or a different type as that used initially, where the population of cells can be mitotically expanded. The cells of the invention may be used at any point between passage 0 and senescence. The cells preferably are passaged between about 3 and about 25 times, more preferably are passaged about 4 to about 12 times, and preferably are passaged 10 or 11 times. Cloning and/or subcloning may be performed to confirm that a clonal population of cells has been isolated.

The cells may be cryopreserved. Accordingly, in a one embodiment, umbilical cord arterial tissue-derived cells for autologous transfer (for either the mother or child) may be derived from appropriate postpartum tissues following the birth of a child, then cryopreserved so as to be available in the event they are later needed for transplantation.

Accordingly, in one embodiment, the invention provides for methods of generating human umbilical cord arterial tissue-derived cells from human umbilical cord tissue. The methods require use of human umbilical cord arterial tissue substantially free of blood (or free of blood). This human umbilical cord arterial tissue substantially free of blood is dissociated by mechanical association such as mincing (or any other process known in the art). The dissociated tissue is then digested with a mixture of enzymes comprising a metalloprotease, neutral protease and mucolytic enzyme. For example, the tissue may be digested for one or more house such as a period of about 0.5 hours to about 2 hours. The digestion step may also include incubating the dissociated tissue with the mixture of enzymes at about 37° C. In certain embodiments, the metalloprotease enzyme is collagenase, the mucolytic enzyme is hyaluronidase and the neutral protease enzyme is dispase. In more preferred embodiments, the dissociated tissue is digested using a combination of hyaluronidase, dispase and collagenase. After digestion, the human umbilical cord arterial tissue-derived cells are isolated from the digested tissue and cultured to obtain a homogenous population of isolated umbilical cord arterial tissue-derived cells. For example, the cells may be cultures for about 10 to about 100 hours, alternatively from about 12 to about 72 hours, alternatively from about 24 hours to about 48 hours, alternatively from about 12 to about 96 hours, alternatively from about 10, 11, 12, 13, 14, 15 hours to about 24, 48, 72 or 96 hours. In one embodiment, the culturing includes selecting for cells that grow and express CD10.

The human arterial tissue substantially free of blood (or free of blood) may be generated by a variety of ways. In certain embodiments, the human arterial tissue only contains the arterial wall tissue whereby the vessels have been separated from Wharton's Jelly. For example, human umbilical cord tissue may be obtained and substantially all of the blood (or all of the blood) from the tissue may be removed to yield umbilical tissue substantially free of blood (or free of blood). The human umbilical cord arterial tissue can then be separated from the umbilical cord tissue. Alternatively, the human umbilical cord arterial tissue is separated from the human umbilical cord tissue prior to removal of substantially all the blood (or all of the blood). In certain embodiments, all of the blood is removed. The removing step comprises removal of free or clotted blood by one or more of washing, suctioning, blotting, centrifugal separation, or enzymatic removal.

The methods of the invention result in the generation of a homogeneous population of postpartum umbilical cord arterial tissue-derived cells. Homogeneous populations of postpartum umbilical cord arterial tissue-derived cells are preferably free of cells of maternal lineage. Homogeneity of a cell population may be achieved by any method known in the art, for example, by cell sorting (e.g., flow cytometry) or by clonal expansion in accordance with known methods. Thus, preferred homogeneous postpartum umbilical cord arterial tissue-derived cells populations may comprise a clonal cell line of postpartum-derived cells. Such populations are particularly useful when a cell clone with highly desirable functionality has been isolated.

Also provided herein are populations of cells incubated in the presence of one or more factors, or under conditions, that stimulate stem cell differentiation along a desired pathway (e.g., neural, epithelial). Such factors are known in the art and the skilled artisan will appreciate that determination of suitable conditions for differentiation can be accomplished with routine experimentation. Optimization of such conditions can be accomplished by statistical experimental design and analysis, for example response surface methodology allows simultaneous optimization of multiple variables, for example in a biological culture. Presently preferred factors include, but are not limited to factors, such as growth or trophic factors, demethylating agents, co-culture with neural or epithelial lineage cells or culture in neural or epithelial lineage cell-conditioned medium, as well other conditions known in the art to stimulate stem cell differentiation along these pathways (for factors useful in neural differentiation, see, e.g., Lang, K. J. D. et al., 2004, J. Neurosci. Res. 76: 184-192; Johe, K. K. et al., 1996, Genes Devel. 10: 3129-3140; Gottleib, D., 2002, Ann. Rev. Neurosci. 25: 381-407).

### 3. Characteristics of Postpartum Umbilical Cord Arterial Tissue-Derived Cells

The postpartum umbilical cord arterial tissue-derived cells of the invention may be characterized, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR)), protein arrays, protein secretion (e.g., by plasma clotting assay or analysis of PDC-conditioned medium, for example, by Enzyme Linked ImmunoSorbent Assay (ELISA)), mixed lymphocyte reaction (e.g., as measure of stimulation of PBMCs), and/or other methods known in the art.

In particular, the cells may be characterized based on their surface proteins or “markers”, which may be used to provide a profile for the identification of the cell or cell line. In certain embodiments, the methods of the invention result in the isolation of cells that express one or more of (or each of) CD13, CD90, NG2, and HLA-ABC and that do not express one or more of (or each of) CD31, CD34, CD45, CD117, FSP1, and E-cadherin. In other embodiments, the postpartum umbilical cord arterial tissue-derived cells express each of the markers CD10, CD13, CD90, HLA-A, B, C and alpha smooth muscle actin, wherein the cells produce the immunologically-detectable proteins which correspond to the listed markers. The cells may also express NG2. The postpartum umbilical cord arterial tissue-derived cells also do not express or produce proteins corresponding to any of the markers CD31, CD34, CD45, CD117 and HLA DR, DP, DQ. In certain embodiments, the cells also do not express or do not produce HLA-C, E-cadherin, FSP-1 and combinations thereof.

In certain embodiments, the umbilical cord arterial tissue-derived cells possess a normal karyotype, which is maintained as the cells are passaged. Methods for karyotyping are available and known to those of skill in the art.

The postpartum umbilical cord arterial tissue-derived cells may also be capable of expansion in culture, have normal karyotypes, and maintain normal karyotypes with passaging. Presently preferred for use with the invention are cells that do not spontaneously differentiate, for example along neural lines. However, the cells may be characterized by their ability to differentiate into at least neural lines. In certain embodiments, the cells are capable of self-renewal and expansion in culture, have the potential to differentiate into cells of other phenotypes (e.g. neural lines).

In certain embodiments, the postpartum umbilical cord arterial tissue-derived cells may also be characterized by their ability to secrete a trophic factor. In particular, the postpartum umbilical cord arterial tissue-derived cells may be characterized by secretion of trophic factors selected from thrombospondin-1, thrombospondin-2 and thrombospondin-4. In other embodiments, the cells secrete thrombospondin-1, thrombospondin-2 and thrombospondin-4. In addition, the cells of the invention secrete one or more of RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

The postpartum umbilical cord arterial tissue-derived cells may also be characterized by their ability to secrete a bridge molecule. In certain embodiments, the umbilical cord arterial tissue-derived cells secrete one or more bridge molecules.

In preferred embodiments, the cells comprise two or more of the above-listed growth, protein/surface marker production, gene expression or substance-secretion characteristics. More preferred are those cells comprising, three, four, or five or more of the above characteristics. Still more preferred are postpartum umbilical cord arterial tissue-derived cells comprising six, seven, or eight or more of the above characteristics. Still more preferred presently are those cells comprising all of the above characteristics.

In one embodiment of the invention, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express each of the markers CD10, CD13, CD90, HLA-A, B, C and alpha smooth muscle actin and do not express or produce proteins corresponding to any of the markers CD31, CD34, CD45, CD117 and HLA DR, DP, DQ. The cells may also express NG2. In certain embodiments, the cells also do not express or do not produce HLA-C, E-cadherin, or FSP-1. In other embodiments, the cells do not express or produce HLA-C, E-cadherin, FSP-1, and combinations thereof. The cells also secrete one or more of thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

In another embodiment of the invention, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express each of the markers CD10, CD13, CD90, HLA-A, B, C, alpha smooth muscle actin, and NG2, and do not express or produce proteins corresponding to any of the markers CD31, CD34, CD45, CD117 and HLA DR, DP, DQ. In certain embodiments, the cells also do not express or do not produce HLA-C, E-cadherin, FSP-1 and combinations thereof.

In another embodiment of the invention, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express each of the markers CD10, CD13, CD90, HLA-A, B, C, alpha smooth muscle actin, and NG2, and do not express or produce proteins corresponding to any of the markers CD31, CD34, CD45, CD117, E-cadherin, and HLA DR, DP, DQ. In certain embodiments, the cells also do not express or do not produce HLA-C, FSP-1 or both. In other embodiments, the cells secrete one or more of thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

In yet another embodiment of the invention, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express each of the markers CD10, CD13, CD90, HLA-A, B, C, alpha smooth muscle actin and do not express or produce proteins corresponding to any of the markers CD31, CD34, CD45, CD117, HLA-C and HLA DR, DP, DQ. In certain embodiments, the cells also do not express or do not produce E-cadherin, FSP-1 or both. The cells may also secrete one or more of thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1. In certain embodiments, the cells also express NG2; in other embodiments the cells do not express NG2.

In an alternate embodiment of the invention, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express each of the markers CD10, CD13, CD90, HLA-A, B, C, alpha smooth muscle actin, and NG2, and do not express or produce proteins corresponding to any of the markers CD31, CD34, CD45, CD117, HLA-C, E-Cadherin and HLA DR, DP, DQ. In certain embodiments, the cells also do not express or do not produce FSP-1. The cells also secrete one or more of thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

In yet another embodiment of the invention, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express each of the markers CD10, CD13, CD90, HLA-A, B, C, alpha smooth muscle actin, and NG2, and do not express or produce proteins corresponding to any of the markers CD31, CD34, CD45, CD117, HLA-C, E-Cadherin, FSP-1 and HLA DR, DP, DQ. The cells are further characterized by their secretion of one or more of thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

In an alternate embodiment, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express CD13, CD90 and HLA-ABC and that do not express CD31, CD34, CD45, CD117, FSP1, E-cadherin and NG2. These cells also secrete thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

Alternatively, the postpartum umbilical cord arterial tissue-derived cells are isolated from human umbilical cord arterial tissue substantially free of blood (or free of blood), are capable of renewal and expansion into culture, have the potential to differentiate into cells of other phenotypes, express CD13, CD90, NG2, and HLA-ABC and that do not express CD31, CD34, CD45, and CD117, and do not express one or more of FSP1, and E-cadherin. These cells also secrete thrombospondin-1, thrombospondin-2, thrombospondin-4, RTK ligands, BDNF, HDF and soluble VEGFR1. In embodiments, the cells secrete one or more of thrombospondin-1, BDNF, and soluble VEGFR1. In embodiments, the cells secrete thrombospondin-1, BDNF, and soluble VEGFR1.

In certain embodiments, the cells of the invention are able to differentiate into an ocular lineage or phenotype. In other embodiments, the cells are able to differentiate into a neural lineage or phenotype. In alternate embodiments, the cells are able to differentiate into: (1) an ocular lineage or phenotype; and (2) a neural lineage or phenotype.

Preferred cells, when grown in growth medium, are substantially stable with respect to the cell markers produced on their surface, and with respect to the expression pattern of various genes, for example as determined using an Affymetrix GENECHIP. The cells remain substantially constant, for example in their surface marker characteristics over passaging, through multiple population doublings.

One feature of the umbilical cord arterial tissue-derived cells of the invention is that they may be deliberately induced to differentiate into various lineage phenotypes by subjecting them to differentiation-inducing cell culture conditions. Of use in treatment of certain ocular degenerative conditions, the umbilical cord arterial tissue-derived cells of the invention may be induced to differentiate into neural phenotypes using one or more methods known in the art. For instance, as exemplified herein, the cells may be plated on flasks coated with laminin in Neurobasal-A medium (Invitrogen, Carlsbad, Calif.) containing B27 (B27 supplement, Invitrogen), L-glutamine and Penicillin/Streptomycin, the combination of which is referred to herein as Neural Progenitor Expansion (NPE) medium. NPE media may be further supplemented with bFGF and/or EGF. Alternatively, the cells may be induced to differentiate in vitro by: (1) co-culturing the umbilical cord arterial tissue-derived cells of the invention with neural progenitor cells; or (2) growing the umbilical cord arterial tissue-derived cells of the invention in neural progenitor cell-conditioned medium.

Differentiation of the umbilical cord arterial tissue-derived cells of the invention into neural phenotypes may be demonstrated by a bipolar cell morphology with extended processes. The induced cell populations may stain positive for the presence of nestin. Differentiated umbilical cord arterial tissue-derived cells may be assessed by detection of nestin, TuJ1 (BIII tubulin), GFAP, tyrosine hydroxylase, GABA and/or MBP. In some embodiments, umbilical cord arterial tissue-derived cells of the invention have exhibited the ability to form three-dimensional bodies characteristic of neuronal stem cell formation of neurospheres.

### 4. Kits and Banks

In another aspect, the invention provides kits that utilize the cells/cell population of the invention, conditioned medium prepared from the cells, and components and products thereof in various methods for ocular regeneration and repair as described above. Where used for treatment of ocular degenerative conditions, or other scheduled treatment, the kits may include one or more cell populations or conditioned medium, including at least the cells of the invention or conditioned medium derived from the cells, and a pharmaceutically acceptable carrier (liquid, semi-solid or solid). The kits also optionally may include a means of administering the cells and conditioned medium, for example by injection. The kits further may include instructions for use of the cells and conditioned medium. Kits prepared for field hospital use, such as for military use may include full-procedure supplies including tissue scaffolds, surgical sutures, and the like, where the cells or conditioned medium are to be used in conjunction with repair of acute injuries. Kits for assays and in vitro methods as described herein may contain, for example, one or more of: (1) postpartum umbilical cord arterial tissue-derived cells or components thereof, or conditioned medium or other products of postpartum umbilical cord arterial tissue-derived cells; (2) reagents for practicing the in vitro method; (3) other cells or cell populations, as appropriate; and (4) instructions for conducting the in vitro method.

In yet another aspect, the invention also provides for banking of tissues, cells, cell populations, conditioned medium, and cellular components of the invention. As discussed above, the cells and conditioned medium are readily cryopreserved. The invention therefore provides methods of cryopreserving the cells in a bank, wherein the cells are stored frozen and associated with a complete characterization of the cells based on immunological, biochemical and genetic properties of the cells. The frozen cells can be thawed and expanded or used directly for autologous, syngeneic, or allogeneic therapy, depending on the requirements of the procedure and the needs of the patient. Preferably, the information on each cryopreserved sample is stored in a computer, which is searchable based on the requirements of the surgeon, procedure and patient with suitable matches being made based on the characterization of the cells or populations. Preferably, the cells of the invention are grown and expanded to the desired quantity of cells and therapeutic cell compositions are prepared either separately or as co-cultures, in the presence or absence of a matrix or support. While for some applications it may be preferable to use cells freshly prepared, the remainder can be cryopreserved and banked by freezing the cells and entering the information in the computer to associate the computer entry with the samples. Even where it is not necessary to match a source or donor with a recipient of such cells, for immunological purposes, the bank system makes it easy to match, for example, desirable biochemical or genetic properties of the banked cells to the therapeutic needs. Upon matching of the desired properties with a banked sample, the sample is retrieved and prepared for therapeutic use. Cell lysates, ECM or cellular components prepared as described herein may also be cryopreserved or otherwise preserved (e.g., by lyophilization) and banked in accordance with the present invention.

### 5. Uses of Postpartum Umbilical Cord Arterial Tissue-Derived Cells

In certain embodiments of the invention, an effective amount of the postpartum umbilical cord arterial tissue-derived cells of the invention may be used to treat ocular degenerative conditions. For such uses, the cells may be provided as part of pharmaceutical composition. They may also be provided as part of a matrix or implantable device. In certain embodiments, the postpartum umbilical cord arterial tissue-derived cells of the invention may be used to provide trophic factors that are suitable for treating ocular degenerative conditions. In alternate embodiments, conditioned media may be used instead of the cells. In yet another embodiment, an extracellular matrix produced by the cells may be used. The cells or conditioned media may also be used as part of combination therapy.

Examples of other components that may be administered with the cells and conditioned media products produced by the cells include, but are not limited to: (1) other neuroprotective or neurobeneficial drugs; (2) selected extracellular matrix components, such as one or more types of collagen known in the art, and/or growth factors, platelet-rich plasma, and drugs (alternatively, the cells may be genetically engineered to express and produce growth factors); (3) anti-apoptotic agents (e.g., erythropoietin (EPO), EPO mimetibody, thrombopoietin, insulin-like growth factor (IGF)-I, IGF-II, hepatocyte growth factor, caspase inhibitors); (4) anti-inflammatory compounds (e.g., p38 MAP kinase inhibitors, TGF-beta inhibitors, statins, IL-6 and IL-I inhibitors, PEMIROLAST, TRANILAST, REMICADE, SIROLIMUS, and non-steroidal anti-inflammatory drugs (NSAIDS) (such as TEPDXALIN, TOLMETIN, and SUPROFEN); (5) immunosuppressive or immunomodulatory agents, such as calcineurin inhibitors, mTOR inhibitors, antiproliferatives, corticosteroids and various antibodies; (6) antioxidants such as probucol, vitamins C and E, coenzyme Q-10, glutathione, L-cysteine and N-acetylcysteine; and (6) local anesthetics, to name a few.

The present invention is further illustrated, but not limited by, the following examples.

### Example 1

### Derivation of Cells from Postpartum Umbilical Cord Arterial Tissue

This example describes the preparation of postpartum umbilical cord arterial tissue-derived cells from umbilical cord tissues. Postpartum umbilical cords were obtained upon birth of either a full term or pre-term pregnancy.

Umbilical cords were obtained following normal deliveries. The cell isolation protocol was performed aseptically in a laminar flow hood. To remove blood and debris, the cord was washed in phosphate buffered saline in the presence of antimycotic and antibiotic (100 units/milliliter penicillin, 100 micrograms/milliliter streptomycin, 0.25 micrograms/milliliter amphotericin B). After removal of the blood and debris, the umbilical cord arterial tissue was then separated from the other umbilical cord tissue. Specifically, the umbilical cord arterial wall was separated from Wharton's Jelly. The umbilical cord arterial tissue was then mechanically dissociated in 150 cm² tissue culture plates in the presence of 50 milliliters of medium (DMEM-Low glucose or DMEM-High glucose; Invitrogen), until the tissue was minced into a fine pulp. The chopped umbilical cord arterial tissue was then transferred to 50-milliliter conical tubes (approximately 5 grams of tissue per tube).

The tissue was then digested in either DMEM-Low glucose medium containing antimycotic and antibiotic as described above. For the digestion, an enzyme mixture of collagenase, dispase and hyaluronidase was used (collagenase, 500 Units/milliliter; dispase, 50 Units/milliliter; and hyaluronidase (Sigma), 5 Units/milliliter, in DMEM-Low glucose). The conical tubes containing the tissue, medium and digestion enzymes were incubated at 37° C. in an orbital shaker (Environ, Brooklyn, N.Y.) at 225 rpm for 2 hours.

After digestion, the tissue was centrifuged at 150×g for 5 minutes, and the supernatant was aspirated. The pellet was resuspended in 20 milliliters of growth medium (DMEM-Low glucose (Invitrogen), 15 percent (v/v) fetal bovine serum, 0.001% (v/v) 2-mercaptoethanol (Sigma), 1 milliliter per 100 milliliters of antibiotic/antimycotic as described above). The cell suspension was filtered through a 70-micrometer nylon cell strainer (BD Biosciences). An additional 5 milliliters rinse comprising growth medium was passed through the strainer. The cell suspension was then passed through a 40-micrometer nylon cell strainer (BD Biosciences) and chased with a rinse of an additional 5 milliliters of growth medium.

The filtrate was resuspended in growth medium (total volume 50 milliliters) and centrifuged at 150×g for 5 minutes. The supernatant was aspirated and the cells were resuspended in 50 milliliters of fresh growth medium. This process was repeated twice more.

Upon the final centrifugation, supernatant was aspirated and the cell pellet was resuspended in 5 milliliters of fresh growth medium. The number of viable cells was determined using Trypan Blue staining. Cells were then cultured under standard conditions until a homogeneous population of cells was obtained.

The cells isolated from umbilical cord arterial tissue were seeded at 5,000 cells/cm² onto gelatin-coated T-75 cm² flasks (Corning Inc., Corning, N.Y.) in growth medium with antibiotics/antimycotics as described above. After 2 days spent medium was aspirated from the flasks. Cells were washed with PBS three times to remove debris and blood-derived cells. Cells were then replenished with growth medium and allowed to grow to confluence (about 10 days from passage 0) to passage 1. On subsequent passages (from passage 1 to 2 and so on), cells reached sub-confluence (75-85 percent confluence) in 4-5 days. For these subsequent passages, cells were seeded at 5000 cells/cm². Cells were grown in a humidified incubator with 5 percent carbon dioxide and atmospheric oxygen, at 37° C. The cells were passaged until a homogenous population of cells was obtained.

### Example 2

### Comparison of Surface Marker Profiles

As discussed above, human umbilical cord tissue-derived cells (“hUTC”) obtained using the isolation protocol disclosed in U.S. Pat. No. 7,510,873 have been proven to be therapeutically useful in the treatment of various diseases. In particular, most recently, the cells have been found useful in the treatment of ocular degenerative conditions. See e.g. U.S. 2016/0158293, 2016-0166619 and U.S. Ser. No. 15/366,599.

The surface marker profile of hUTC was compared to endothelial cells (EC), umbilical cord blood cells (CBC), fibroblasts (FB), placental epithelial cells (PEC), umbilical smooth muscle cells (USMC) (i.e. the source of cells of the invention) and placental pericyte cells (PC). The results of this comparison are shown in Tables 2-1 and Tables 2-2 below.

TABLE 2-1
hUTC cell identity markers

| | Type of cell | | | | | | |
|---|---|---|---|---|---|---|---|
| | hUTC | EC | CBC | FB | PEC | USMC | PC |
| Markers | (%) | (%) | (%) | (%) | (%) | (%) | (%) |
| CD13 | 92.9 | 99.0 | 94.5 | 98.6 | 56.2 | 99.3 | 97.8 |
| CD90 | 99.4 | 5.4 | 3.2 | 82.9 | 28.4 | 99.7 | 99.3 |
| HLA ABC | 98.7 | 99.7 | 99.6 | 98.8 | 99.8 | 99.2 | 99.8 |
| CD34 | 1.4 | 74.9 | 26.1 | 5.7 | 3.2 | 2.6 | 2.9 |
| CD117 | 3.9 | 43.4 | 6.5 | 4.5 | 1.9 | 1.3 | 6.3 |
| HLA DR, DP, | 0.09 | 0.8 | 25.3 | 8.6 | 0.4 | 0.5 | 2.0 |
| DQ | | | | | | | |

hUTC = human umbilical cord tissue-derived cells
EC = endothelial cells
CBC = umbilical cord blood cells
FB = fibroblasts
PEC = placental epithelial cells
USMC = umbilical smooth muscle cells
PC = placental pericyte cells

TABLE 2-2
Other cell type markers

| | Type of cell | | | | | | |
|---|---|---|---|---|---|---|---|
| | hUTC | EC | CBC | FB | PEC | USMC | PC |
| Markers | (%) | (%) | (%) | (%) | (%) | (%) | (%) |
| CD31 | 0.6 | 96.3 | 92.3 | 1.8 | 1.2 | 0.9 | 1.3 |
| CD45 | 1.7 | 0.9 | 99.6 | 1.9 | 1.7 | 1.0 | 2.4 |
| E-Cadherin | 3.6 | 4.0 | 20.5 | 5.3 | 94.8 | 3.4 | 43.1 |
| FSP-1 | 12.9 | 8.5 | 46.0 | 87.7 | 82.7 | 10.7 | 17.1 |
| α-smooth muscle | 56.2 | 9.2 | 6.6 | 13.0 | 21.6 | 99.7 | 48.4 |
| actin | | | | | | | |
| NG2 | 6.9 | 22.8 | 69.8 | 61.2 | 66.3 | 79.9 | 99.3 |
| CD10 | 79.3 | | | | | 98.2 | |
| HLA-C | 0.6 | | | | | 2.8 | |
| HAPLN1 | 1.7 | | | | | | |
| SYNCAM | 3.6 | | | | | | |

hUTC = human umbilical cord tissue-derived cells
EC = endothelial cells
CBC = umbilical cord blood cells
FB = fibroblasts
PEC = placental epithelial cells
USMC = umbilical smooth muscle cells
PC = placental pericyte cells

Aortic smooth muscle cells were also tested for HLA-C (2.0%), HAPLN1 (99.8%) and SYNCAM (62.9%).

### Example 3

### Characterization of Postpartum Umbilical Cord Arterial Smooth Muscle Tissue-Derived Cells

The postpartum umbilical cord arterial tissue-derived cells were analyzed for their surface markers. Specifically, the cells were tested for the presence or absence of CD13, CD90, NG2, HLA ABC, CD34, CD117, HLA DR, DP, DQ, CD31, CD45, E-Cadherin, FSP1, CD10, HLA-C, and α-smooth muscle actin. The postpartum umbilical cord arterial tissue-derived cells were found to be positive for CD13, CD90, NG2, HLA-ABC, α-smooth muscle actin and CD10. The postpartum umbilical cord arterial tissue-derived cells were also found to be negative for CD31, CD34, CD45, CD117, HLA-DR, DP, DQ, HLA-C, and E-Cadeherin.

### Example 4

### Screening of Postpartum Umbilical Cord Arterial Smooth Muscle Tissue-Derived Cells for Trophic Factor Secretion

The secretion of selected trophic factors from umbilical cord arterial tissue-derived cells was evaluated. In particular, the umbilical cord arterial tissue-derived cells were tested for their ability to secrete a synaptogenic factor or a bridge molecule. The trophic factor secretion from umbilical cord arterial tissue-derived cells (umbilical arterial smooth muscle cells; UASMC) was compared to trophic factor secretion from hUTC (Example 2 above).

Briefly, the cells were cultured under various conditions to allow secretion of the trophic factors in the medium. The cells were cultured in two media: the first (media 1) was DMEM-low glucose supplemented with 15% (v/v) fetal bovine serum and 4 mM L-glutamine; and the second (media 2) was Clonetics™ smooth muscle basal medium (Lonza, Inc.) containing 5% FBS. After culturing, the medium was collected by centrifugation at 14,000×g for 5 minutes and stored at −20° C. The number of cells in each and population doubling level (PDL) were determined, using Vi-CELL XR 2.04 (Beckman Coulter, Inc.).

The cells were tested for brain-derived neurotrophic factor (BDNF), thrombospondin-1 (TSP-1), and soluble vascular endothelial growth factor receptor 1 (sVEGFR1). The amount of each trophic factor was determined by ELISA assay (performed according to the manufacturer's instructions). The amount of each trophic factor can also be determined using SEARCHLIGHT Multiplexed ELISA.

The results of the trophic factor secretion and comparison are shown in Table 4-1 below. UASMC secretes BDNF, TSP-1 and sVEGFR1 in both media 1 and 2. TSP-1 expression was significantly higher in UASMC compared to hUTC.

TABLE 4-1

| | hUTC | UASMC | hUTC | UASMC |
|---|---|---|---|---|
| | Media 1 | Media 1 | Media 2 | Media 2 |
| PDL | 2.7 | 1.6 | 2.5 | 1.6 |
| PDL hrs | 27.4 | 45.5 | 28.7 | 46 |
| BDNF | 475.5 | 579.3 | 202.9 | 373.4 |
| pg/mL/million cells | | | | |
| TSP1 | 119.7 | 502 | 77.8 | 218.5 |
| ng/mL/million cells | | | | |
| sVEGFR1 | 1077.7 | 852.7 | 588.5 | 513.7 |
| pg/mL/million cells | | | | |

Various patents and other publications are referred to throughout the specification. Each of these publications is incorporated by reference herein, in its entirety.

Although the various aspects of the invention have been illustrated above by reference to examples and preferred embodiments, it will be appreciated that the scope of the invention is defined not by the foregoing description but by the following claims properly construed under principles of patent law.

What is claimed is:

1. A method for generating human umbilical cord arterial tissue-derived cells from human umbilical cord tissue comprising the steps of:
(a) obtaining human umbilical cord arterial tissue;
(b) removing substantially all of the blood from the tissue to yield umbilical tissue substantially free of blood;
(c) dissociating the human umbilical cord arterial tissue substantially free of blood by mechanical dissociation such as mincing;
(d) digesting the dissociated tissue with a mixture of enzymes comprising a metalloprotease, neutral protease and mucolytic enzyme;
(e) isolating the umbilical cord arterial tissue-derived cells from the digested tissue; and
(f) culturing the cells to obtain a homogenous population of isolated umbilical cord arterial tissue-derived cells,
wherein the cells are capable of self-renewal and expansion in culture, have the potential to differentiate into cells of other phenotypes and further have the following characteristics:
express CD13, CD90, NG2, and HLA-ABC;
do not express CD31, CD34, CD45, CD117; and
do not express FSP1 or E-cadherin.

2. The method of claim 1, wherein the method further comprises suspending the cells in a growth medium after isolation.

3. The method of claim 1, wherein the method comprises culturing the cells for about 10 to about 100 hours to obtain a homogenous population of isolated umbilical cord arterial tissue-derived cells.

4. The method of claim 1, wherein the culturing comprises selecting for cells that grow and express CD10.

5. The method of claim 1, wherein the removing step comprises removal of free or clotted blood by one or more of washing, suctioning, blotting, centrifugal separation, or enzymatic removal.

6. The method of claim 1, wherein the metalloprotease enzyme is collagenase.

7. The method of claim 1, wherein the neutral protease enzyme is dispase.

8. The method of claim 1, wherein the mucolytic enzyme is hyaluronidase.

9. The method of claim 1, wherein the digestion step comprises incubating the dissociated tissue with the mixture of enzymes at about 37° C.

10. The method of claim 1, wherein the digestion step comprises incubating the dissociated tissue with the mixture of enzymes for one or more hours.

11. The method of claim 1, wherein method comprises digesting the dissociated tissue with a mixture of collagenase, dispase and hyaluronidase.

12. The method of claim 1, further comprising expanding the population of isolated umbilical cord arterial tissue-derived cells in culture for about 10 days to confluence.

13. The method of claim 1, wherein the cells do not express CD31, CD34, CD45, CD117, FSP1, E-cadherin.

14. The method of claim 1, wherein the cells secrete a trophic factor suitable for treating ocular degeneration.

15. The method of claim 1, wherein the cells secrete one or more of thrombospondin-1, BDNF, and soluble VEGFR1.

16. The method of claim 1, wherein the cells have increased expression of TSP-1 compared to umbilical cord tissue-derived cells.

17. The method of claim 1, wherein the cells are suitable for treating ocular degenerative conditions.

18. The method of claim 1, wherein the step of dissociating comprises mincing the human umbilical cord arterial tissue.

19. The method of claim 1, wherein the step of obtaining comprises separating umbilical cord arterial tissue from Wharton's Jelly.

20. The method of claim 1, wherein the umbilical cord arterial tissue-derived cells are umbilical cord arterial smooth muscle tissue-derived cells.

21. The method of claim 1, wherein the step of culturing comprises passaging the cells under conditions sufficient to generate a homogenous population of isolated umbilical cord arterial tissue-derived cells.

22. A method of treating an ocular degenerative condition comprising administering to a subject a homogenous population of isolated umbilical cord arterial tissue-derived cells obtained from human umbilical cord arterial tissue substantially free of blood, wherein the umbilical cord arterial tissue-derived cells express CD13, CD90, NG2, and HLA-ABC, and do not express CD31, CD34, CD45, or CD117.

23. The method of claim 22, wherein the cells secrete one or more of thrombospondin-1, BDNF, and soluble VEGFR1.

24. The method of claim 22, wherein the cells have increased expression of TSP-1 compared to umbilical cord tissue-derived cells.
